(19) United States
(12) Patent Application Publication
(10) Pub. No.: US 2002/0002146 A1
(43) Pub. Date: Jan. 3, 2002

(54) Compositions and methods for the production of S-adenosylmethionine within the body

(76) Inventor: Brian D. Halevie-Goldman, Creek, CA (US)

Correspondence Address: Mr. Richard H. Zaitlen, PILLSBURY WINTHROP LLP, Suite 2800, 725 South Figueroa Street, Los Angeles, CA, 90017, US

(21) Appl. No.: 09/781,822

(22) Filed: Feb. 12, 2001

Related U.S. Application Data
(60) Provisional application No. 60/181,799, filed on Feb. 11, 2000.

(51) Int. Cl. A61K 31/7105; A61K 31/714; A61K 31/198; A61K 31/19

(52) U.S. Cl. 514/47; 514/52; 514/562; 514/561; 514/563; 514/574

(57) ABSTRACT

Described herein is a method for increasing levels of S-adenosylmethionine within the human body without administering S-adenosylmethionine directly. The method of the invention may be achieved by administering one or more of L-methionine, betaine, and malic acid, together with at least one compound selected from the group consisting of folic acid, vitamin B₁₂, magnesium, calcium, and other cofactors.

This application claims the benefit of priority under 35 U.S.C. § 119 of provisional application Ser. No. 60/181,799, filed Feb. 11, 2000, the contents of which are hereby incorporated by reference.

### FIELD OF THE INVENTION

Embodiments of the present invention are directed to promoting the healthful effects of S-adenosylmethionine (“SAMe,” pronounced “sammy”) within the human body or other mammalian host without directly administering SAMe.

### BACKGROUND OF THE INVENTION

SAMe is an important naturally-occurring substance in mammals. It plays an essential role in regulating the cell and the various biochemical processes that occur within it, from the expression of genes to the action of hormones and neurotransmitters.

SAMe is the principal methyl donor in mammals. The process of methylation is essential to building molecules and controlling the reactions between them. Most organic molecules are built along a carbon skeleton comprised of chains, rings, or other conformations of carbon atoms. The “vertebra” of this skeleton is an atom of carbon attached to three hydrogen atoms, forming a methyl group (CH₃). When organic molecules are assembled, it takes a certain amount of energy to attach a methyl group to a growing carbon chain. SAMe provides this energy. It contains within its structure the capacity to catalyze the transfer of single carbon or methyl groups in the most energy efficient manner. This capacity conferred survivability to the earliest self-organizing life forms and has made SAMe an important methyl donor in virtually every living organism on Earth, from single-cell prokaryotes to higher primates and human beings.

SAMe is the sulfonium form of the condensation of the high-energy compound ATP (adenosine triphosphate) and the essential amino acid methionine. Its structure is set forth below in Formula I:

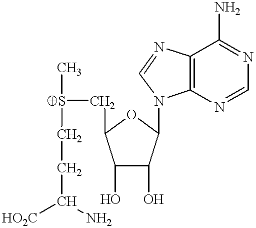

SAMe is formed within the body from methionine and ATP in a reaction catalyzed by methionine adenosyl transferase. The presence of the sulfonium ion activates the methyl group adjacent to it. This methyl group may be transferred to the amino and hydroxy acceptors of a variety of molecules, such as guanidoacetate to yield creatine, ribosomal and transfer RNA to yield methylated RNA, and norepinephrine to yield epinephrine. In addition to such transmethylation reactions, SAMe plays an important role in transsulphuration and transaminopropylation reactions, as well. For example, SAMe is a substrate of a specific lyase that converts the lyase to methylthioadenosine (MTA) and homoserine; it can be an aminoacidic chain donor in the biosynthesis of biotin; it can be a donor of the adenosyl moiety; it can be a promoter of lysin-1,3-amino mutase, threonine synthetase, pyruvate formate lyase, and N5-methyltetrahydrofolate-homocysteine methyltransferase; it can be an inhibitor of H ribonuclease, methylene tetrahydrofolic reductase, and ethanolaminephosphate cytidyltransferase; it is important for bacterial and leukocyte chemotaxis; and it is required in the prokaryote and eukaryote restriction and modification system of DNA. U.S. Pat. No. 6,020,139 (“the '139 patent”), the disclosure of which is incorporated herein by reference, describes additional biochemical pathways by which SAMe is metabolized in the body.

On a broader level, SAMe regulates gene expression and helps prevent genetic mutations; it maintains mitochondrial function; it participates in phospholipid synthesis and maintains the integrity of cell membranes; and it regulates neurotransmitters such as serotonin and melatonin, and hormones such as dopamine and adrenaline

Administering SAMe to subjects has been found to have a variety of salutary effects. U.S. Pat. No. 5,166,328, the disclosure of which is incorporated by reference, describes some of these effects in the brain: it inhibits neuron death following ischemia; it improves the utilization of glucose in the brain; it inhibits brain edema; it improves EEG; it improves evoked potential; and it improves motor function, such as that impaired by stroke. SAMe has been found to enhance emotional well-being and is as effective as many common prescription drugs—tricyclics such as Elavil® (amitriptyline HCl) and Norpramin® (desipramine hydrochloride), and Selective Serotonin Reuptake Inhibitors (SSRIs) such as Prozac® (fluoxetine hydrochloride), Zoloft® (sertraline hydrochloride), and Paxil® (paroxetine hydrochloride)—in treating depression, but with significantly fewer side effects than any of these drugs. SAMe has also been used to treat anxiety, chronic pain, rheumatoid fibromyalgia, Chronic Fatigue Syndrome, cognitive difficulties associated with Alzheimer's Disease, and neurovascular disease. In addition to diseases of the central and peripheral nervous system, SAMe has been found to improve diseases of the joints, cardiovascular system, and liver.

Current SAMe therapy has serious shortcomings. SAMe is expensive. Preparations of SAMe cost (as of early 2001) anywhere from $1.00 to $2.50 for a single 200 mg dose. Such a dose, moreover, can benefit most subjects only mildly; treating depression, neurodegenerative disorders, and other serious conditions can require a dose of 1,600 mg or more, one to three times a day, making long-term treatment too expensive for most consumers.

SAMe is difficult to store. It is highly reactive and very hygroscopic; moisture or heat quickly degrade it. At 35° C. (95° F.), for example, SAMe will remain stable for only 8-10 hours. Making a stable SAMe salt, with tosylate, disulfate tosylate, or 1,4-butanedisulfonate, for example, increases manufacturing cost and partially accounts for the high cost of SAMe.

SAMe is difficult to administer. In most cases, SAMe is administered orally. It is sold as an over-the-counter preparation, making administration via other routes, such as by injection, suppository, or other parenteral routes, impractical or undesirable. When administered orally, some of the SAMe is consumed by intestinal flora, some of which may be pathogenic bacteria. Poor absorption of SAMe in the stomach and in the body requires the administration of large doses of SAMe to achieve the intended effect. Because SAMe is expensive, this is a serious shortcoming. As a result, much of the SAMe administered orally does not enter the bloodstream, and pharmacokinetic studies have failed to show that exogenous SAMe enters the intracellular compartment intact. SAMe contains several water-soluble groups, such as hydroxyl groups, amino groups, a sulfonium group, and a carboxyl group, and as a result has only a weak tendency to cross the lipid-rich membrane of the cell.

Administering SAMe orally can lead to serious side effects in certain individuals. SAMe can create or exacerbate an over-methylated state, leading to a manic state in individuals suffering from bipolar disorder, for example. SAMe donates a methyl group to become S-adenosyl-L-homocysteine; a hydrolase then cleaves this molecule, yielding adenosine and L-homocysteine. High levels of homocysteine have been linked to cardiovascular and neurovascular disease, and can be dangerous for individuals with high blood pressure and angina. In many susceptible individuals, administering SAMe orally may disturb the body's natural regulation of these reactions, resulting in elevated levels of homocysteine.

### SUMMARY OF THE INVENTION

It is an object of the invention to achieve the beneficial effects of SAMe without the problems associated with its direct oral administration. It is a further object of the invention to provide a composition that the body can readily absorb, that is easy to manufacture, and that can increase the effectiveness of SAMe therapy. It is a further object of the invention to provide a method of achieving the beneficial effects of SAMe at less than the current high cost of SAMe therapy, thereby enabling a greater number of consumers to benefit from its effects. Still another object of the invention is to achieve the beneficial effects of SAMe without excessively elevating levels of homocysteine within the body.

Described herein is a method of increasing levels of SAMe within the body and achieving its beneficial effects without administering SAMe directly. The method of the invention comprises administering to subject one or more of L-methionine, betaine, and malic acid, together with at least one compound selected from the group consisting of folic acid, vitamin B₁₂, magnesium, calcium, and other cofactors.

It is a remarkable feature of the invention that one can obtain, with natural ingredients, therapeutic benefits equivalent to or greater than SAMe without administering SAMe directly, a discovery hitherto unknown in the art. The method of the invention is inexpensive, as the compositions required to perform it are inexpensive and widely available commercially. The method of the invention is moreover safer than administering SAMe directly, because increases intracellular SAMe to levels required to achieve therapeutic effect without increasing homocysteine levels.

### DETAILED DESCRIPTION OF THE INVENTION

The method of the invention may be achieved by administering one or more of L-methionine, betaine, and malic acid, together with at least one compound selected from the group consisting of folic acid, vitamin B₁₂, magnesium, calcium, and other cofactors. All of these compounds are inexpensive and readily available from a wide variety of commercial sources.

In a first embodiment of the invention, the method comprises administering to a subject a formulation comprising L-methionine together with a compound selected from the group consisting of betaine (also known as TMG or trimethylglycine), malic acid (or malate), folic acid (or folate), vitamin B₁₂ and other co-factors.

In a second embodiment of the invention, the method comprises administering to a subject a formulation comprising betaine and a compound selected from the group consisting of L-methionine, malic acid, folic acid, vitamin B₁₂ and other co-factors.

In a third embodiment of the invention, the method comprises administering to a subject a formulation comprising malic acid (or malate), and a compound selected from the group consisting of L-methionine, betaine, folic acid (or folate), vitamin B₁₂ and other co-factors.

As used herein, the term “co-factors” refers to those compounds which are important in the methylation process. They are set forth below in Table 1, along with the doses at which they may be used with the present invention.

| | PREFERRED | |
|---|---|---|
| COFACTOR | DOSE | DOSE |
| Pyridoxine (Vitamin B6) | 10 mg | 02 mg-1,000 mg |
| Calcium Citrate | 120 mg | 0-500 mg |
| Folate or folic acid/day | 800 mcg | 0-1,000 mcg |
| Folate as Methyl | 600 mcg | 0-1,000 mcg |
| tetahydrofolate/day | | |
| Alpha-lipoic acid | 2 mg | 0-5 mg |
| Choline Bitartrate | 500 mg | 0-1,000 mg |
| Vitamin B12 as | 300 mcg | 0-500 mcg |
| Methylcobalamin | | |
| Thiamine (Vitamin B1) | 1.5 mg | 0-2 mg |
| L-Serine | 50 mg | 0-300 mg |
| Manganese Picolinate | 1.5 mg | 0.0-2 mg |
| Riboflavin (Vitamin B2) | 10 mg | 0-10 mg |
| Biotin | 200 mcg | 0-400 mcg |
| Bioflavenoids | 100 mg | 0-500 mg |
| (e.g. Quercetin) | | |
| Vitamin C | 60 mg | 0-600 mg |
| Chromium as | 200 mcg | 0-200 mcg |
| GTF-chromium | | |
| Choline Bitartrate | 250 mg | 0-1,500 mg |
| Vitamin E Succinate | 800 IU | 0 IU-1,200 IU |
| (alpha tocopherol) | | |
| Vitamin E | 800 IU | 0 IU-1,200 IU |
| (mixed tocopherols, e.g., | | |
| gamma-tocopherol) | | |
| L-Glutamine | 500 mg | 0-1,000 mg |
| N-acetyl-cysteine | 50 mg | 0-200 mg |
| Intrinsic Factor | 10 mg | 0-100 mg |
| Vitamin B3 as niacin | 20 mg | 0-200 mg |
| or nicotinic acid | | |
| Vitamin B3 as | 20 mg | 0-200 mg |
| niacinamide | | |
| CoEnzyme Q10 | 25 mg | 0-40 mg |
| L-Lysine | 100 mg | 0-500 mg |
| L-Threonine | 500 mg | 150-2000 |
| Selenomethionine or | 150 mcg | 50-300 mcg |
| kelp extract | | |
| Zinc Methionate or | 15 mg | 5-20 mg |
| Monomethionine | | |

All doses stated above are expressed as daily doses. Doses for the principal active active ingredients are set forth below in Table 2.

| | PREFERRED | |
|---|---|---|
| COMPOUND | DOSE | DOSE |
| L-Methionine | 500 mg | 0-10,000 mg |
| Magnesium methionate | 500 mg | 0-10,000 mg |
| or Magnesium | | |
| monomethioninate | | |
| Magnesium aspartate | 180 mg | 0-5,000 mg |
| Magnesium malate | 500 mg | 0-10,000 mg |
| Malate or malic acid | 800 mg | 0-10,000 mg |
| betaine | 1,000 mg | 0-10,000 mg |

As with Table 1, all doses are expressed as daily doses. In one presently preferred embodiment, the principal active ingredients of table 2 and the co-factors of Table 1 are administered at a dose of approximately 3,000 mg total principal active ingredients and co-factors a day. This dose is preferably administered in two equivalent doses per day (that is, in two 1,500 mg doses).

The foregoing ingredients are those that promote the production of either (1) ATP; (2) L-methionine; (3) methyl-donors that re-methylate methyl-acceptors back into methyl donors and diminish the depletion of SAMe; (4) various essential nutritional co-factors (e.g., vitamins and minerals) that are important for methionine metabolism and formation of ATP but are often deficient in the diets of many individuals; and (5) ingredients that are likely to reduce the chance of a build-up of the metabolic waste product homocysteine. These combined effect of these ingredients broaden the spectrum of conditions for which SAMe is ordinarily indicated.

While these elements may be provided to the subject through diet, the subject may also ingest them in pill, powder or liquid form. It is desirable that the methylation cofactors, particularly folate (vitamin B11), Vitamin B12 and trimethylglycine (TMG), are present. As TMG is converted to dimethyl glycine (DMG), it shifts the metabolic current away from homocysteine.

Administering L-methionine with TMG or betaine allows intestinal flora and non-target organ systems to preferentially consume TMG or betaine rather than L-methionine, thereby allowing a greater proportion of L-methionine to remain for use by target organ systems. TMG and betaine also help prevent the conversion of methionine to homocysteine, thereby preventing homocysteine levels from becoming elevated.

Another formulation for use in the method of the invention comprises L-glutamine and L-methionine as the principal active ingredients. As with TMG and betaine, intestinal flora and non-target organ systems preferentially consume L-glutamine over L-methionine, thereby preserving L-methionine for absorption into the blood stream where it is transported to target organ systems. L-glutamine is converted in the body into glutamate, a precursor of γ-aminobutyric acid (GABA). GABA is the principal inhibitory neurotransmitter in the brain; administering it inhibits central nervous system activity and therefore has a calming effect.

Another formulation for use in the method of the invention comprises ATP and L-methionine as the principal active ingredients. L-methionine in combination with ATP aids in the intracellular conversion of L-methionine into SAMe.

Formulations for use in the method of the invention may additionally contain vitamin B11 (folic acid) and vitamin B12 (cobalamin), to aid in the metabolism of homocysteine to methionine.

L-glyceine may also be added to the invention to facillitate methylation.

Calcium and magnesium aids in converting L-methionine into SAMe. They are preferably provided in a ratio of about two parts calcium to one part magnesium.

N-acetylcysteine also aids in converting L-methionine into SAMe, and additionally protects against a toxic build-up of homocysteine.

The method of the present invention may be used to treat any condition for which SAMe is indicated. It is particularly effective in the treatment and prevention of conditions contrary to good mental health, especially depression. As used herein, “conditions contrary to good mental health” include any psychological or organic condition that impairs normal functioning. Examples of such conditions include, but are not limited to, somatoform disorders, such as conversion disorder, hypochondria, and body dysmorphic disorder; anxiety disorders, such panic disorder, phobias, obsessive compulsive disorder, and acute stress disorder; dissassociative disorders, such as dissociative amnesia, multiple personality disorder, and depersonalization disorder; mood disorders, such as depression, dysthymic disorder, bipolar disorder (bipolar I and bipolar II disorders), cyclothymic disorder; personality disorders, such as paranoia, schizoid and schizotypal personalities, borderline personality, antisocial personality, narcissistic personality, histrionic personality, dependent personality, and obsessive-compulsive personality; psychosexual disorders, such as hypoactive sexual desire disorder and sexual aversion disorder; and schizophrenia and disorders related to it such as delusional disorder.

A condition need not be the kind that requires medical intervention to be considered a “condition contrary to good mental health.” Depression, for example, encompasses major depressive disorder requiring aggressive treatment with antidepressant medications; it also encompasses a mild case of gloominess or “feeling blue” in response to a common stressor, such as parting with a loved one for a weekend, receiving a poor grade on an exam, or even cloudy weather. The method of the invention may be used to treat any of the foregoing conditions.

When used to treat conditions contrary to good mental health, it may be desirable, depending on the condition, to add kava kava root or extracts thereof to the formulations of the invention. Kava kava, the common name for Piper methysticum, is known for its calming effects and is used to treat anxiety. Kava pyrones may be supplied as the cut or dry root of the plant, as a fluid extract, or in any of the other forms well known in the art.

The method of the invention may also be used to treat and prevent liver dysfunction. The term “liver dysfunction,” as used herein, refers to any condition which impairs normal functioning of the liver. It includes, but is not limited to, conditions such as hepatomegaly, portal hypertension, portal-systemic encephalopathy, hepatic steatosis, fibrosis, cirrhosis, hepatitis, hepatocellular necrosis, hepatic granulomas, hepatic cysts, and tumors of the liver, such as hepatocellular adenoma. The method of the invention may be used to treat liver dysfunction and conditions secondary to it, such as jaundice, disorders of bilirubin metabolism, and cholelithiasis.

The method of the invention may also be used to treat diseases of the joints. As used herein, “diseases of the joints” refers to any conditions which impair the normal functioning of the joints, including rheumatoid arthritis, polychondritis, systemic lupus erythematosus, connective tissue disease, ankylosing spondylitis, gout, fibromyalgia, and back pain. The method of the invention is not limited to treating those conditions for which medical intervention is necessary, but may also be used to treat anything from mild swelling of the joints to a sore back.

The method of the invention may also be used to prevent tooth loss, facilitate lactation in pregnant women, to decrease menopausally-related sleep disturbances and other forms of insomnia, to improve the performance of athletic athletes, to improve memory, treat migraines, to treat neurodegenerative diseases such as multiple sclerosis (by repairing myelin), alleviate caffeine craving, promote the healing of ulcers, increase the effectiveness of cold medications (including herbal preparations such as Echinacea). The method may also be used to increase the effectiveness of prescription antidepressants.

In addition to the active ingredients described herein, formulations according to the invention may optionally contain one or more excipients, including the following: preservatives, such as ethyl-p-hydroxybenzoate; suspending agents such as methyl cellulose, tragacanth, and sodium alginate; wetting agents such as lecithin, polyoxyethylene stearate, and polyoxyethylene sorbitan mono-oleate; granulating and disintegrating agents such as starch and alginic acid; binding agents such as starch, gelatin, and acacia; lubricating agents such as magnesium stearate, stearic acid, and talc; and flavoring and coloring agents.

Formulations of the present invention suitable for oral administration may be presented in any of the following forms: discrete units such as capsules, cachets, or tablets each containing a predetermined amount of the active ingredient; powder or granules; solutions or suspensions in an aqueous liquid or a non-aqueous liquid; or, as oil-in-water liquid emulsions or water-in-oil emulsions, and any other form suitable for oral administration.

In one alternative embodiment, the formulation is contained within a food stuff, such as a cookie, a bar of chocolate, a gum or jelly (e.g., a gummy bear), yogurt. The formulation may be added to an egg mix to counteract the high content of cholesterol and saturated fats found in eggs. The formulation may also be added to coffee creamer (for use with decaffeinated coffee or tea) to relieve caffeine craving and to mitigate the effects of aluminum silicate commonly found in creamers.

In still further embodiments, the formulation may be added to an antacid preparation such as those containing sodium bicarbonate, aluminum hydroxide, or magnesium hydroxide. The formulation promotes methylation of DNA in the stomach, prevents stomach and gastrointestinal cancers, and promotes the healing of ulcers.

Formulations for rectal administration may be presented as a suppository with a suitable base comprising, for example, cocoa butter or a salicylate.

Formulations suitable for nasal administration may comprise either solid or liquid preparations. Where the carrier is a solid, it may be a coarse powder having a particle size, for example, in the range of 20 to 500 microns. This powder is administered in the manner in which snuff is taken, i.e., by rapid inhalation through the nasal passage from a container of the powder held close up to the nose. Where the carrier is a liquid, it may be administered as a nasal spray or as nasal drops.

Formulations suitable for vaginal administration may be presented as pessaries, tampons, creams, gels, pastes, foams, or spray formulations.

Formulations suitable for parenteral administration include, for example, aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. The formulations may be presented, for example, in unit-dose or multi-dose containers, sealed ampules and vials, and may be stored in freeze-dried (lyophilized) conditions requiring only the addition of the sterile liquid carrier immediately prior to use.

While the invention has been described in connection with specific exemplary embodiments, it will be apparent to those skilled in the art that various changes can be made to the structure, arrangement, proportions, elements, and materials used in the practice of the invention without departing from its principles.

What is claimed is:

1. A method of treating and preventing conditions contrary to good mental health by administering to a subject a formulation comprising L-methionine and a compound selected from the group consisting of betaine, trimethylglycine, L-glutamine, adenosine triphosphate, and malic acid.

2. The method of claim 1, wherein the formulation further comprises N-acetylcysteine.

3. The method of claim 1, wherein the formulation further comprises at least one B-vitamin selected from the group consisting of vitamin B11 and vitamin B12.

4. The method of claim 2, wherein the formulation further comprises at least one B-vitamin selected from the group consisting of vitamin B11 and vitamin B12.

5. The method of claim 1, wherein the formulation further comprises calcium and magnesium.

6. The method of claim 5, wherein the calcium and magnesium are provided in a ratio of approximately 2 to 1 (2:1) of calcium to magnesium.

7. The method of claim 2, wherein the formulation further comprises calcium and magnesium.

8. The method of claim 7, wherein the calcium and magnesium are provided in a ratio of approximately 2 to 1 (2:1) of calcium to magnesium.

9. The method of claim 3, wherein the formulation further comprises calcium and magnesium.

10. The method of claim 9, wherein the calcium and magnesium are provided in a ratio of approximately 2 to 1 (2:1) of calcium to magnesium.

11. The method of claim 4, wherein the formulation further comprises calcium and magnesium.

12. The method of claim 11, wherein the calcium and magnesium are provided in a ratio of approximately 2 to 1 (2:1) of calcium to magnesium.

13. The method of claim 1, wherein the formulation further comprises a supplement selected from the group consisting of zinc, magnesium, and vitamin B6.

14. The method of claim 1, wherein the formulation further comprises kava kava.

15. The method of claim 1, wherein the compound is L-glutamine.

16. The method of claim 15, wherein the formulation further comprises kava kava.

17. The method of claim 1, wherein the condition is depression.

18. A method of treating liver dysfunction and maintaining the health of the liver, the method comprising administering to a subject a formulation comprising L-methionine and a compound selected from the group consisting of betaine, trimethylglycine, L-glutamine, adenosine triphosphate, and malic acid.

19. The method of claim 18, wherein the formulation further comprises N-acetylcystein.

20. The method of claim 18, wherein the formulation further comprises at least one B-vitamin selected from the group consisting of vitamin B11 and vitamin B12.

21. The method of claim 19, wherein the formulation further comprises at least one B-vitamin selected from the group consisting of vitamin B11 and vitamin B12.

22. The method of claim 18, wherein the formulation further comprises calcium and magnesium.

23. The method of claim 22, wherein the calcium and magnesium are provided in a ratio of approximately 2 to 1 (2:1) of calcium to magnesium.

24. The method of claim 19, wherein the formulation further comprises calcium and magnesium.

25. The method of claim 24, wherein the calcium and magnesium are provided in a ratio of approximately 2 to 1 (2:1) of calcium to magnesium.

26. The method of claim 20, wherein the formulation further comprises calcium and magnesium.

27. The method of claim 26, wherein the calcium and magnesium are provided in a ratio of approximately 2 to 1 (2:1) of calcium to magnesium.

28. The method of claim 21, wherein the formulation further comprises calcium and magnesium.

29. The method of claim 28, wherein the calcium and magnesium are provided in a ratio of approximately 2 to 1 (2:1) of calcium to magnesium.

30. The method of claim 18, wherein the formulation further comprises a supplement selected from the group consisting of zinc, magnesium, and vitamin B6.

31. The method of claim 18, wherein the formulation further comprises kava kava.

32. The method of claim 18, wherein the compound is L-glutamine.

33. The method of claim 32, wherein the formulation further comprises kava kava.

34. The method of claim 18, wherein the dysfunction is selected from the group consisting of cirrhosis, hepatitis, and jaundice.

35. A method of treating and preventing diseases of the joints, the method comprising administering to a subject a formulation comprising L-methionine and a compound selected from the group consisting of betaine, trimethylglycine, L-glutamine, and adenosine triphosphate.

36. The method of claim 35, wherein the formulation further comprises N-acetylcystein.

37. The method of claim 35, wherein the formulation further comprises at least one B-vitamin selected from the group consisting of vitamin B11 and vitamin B12.

38. The method of claim 36, wherein the formulation further comprises at least one B-vitamin selected from the group consisting of vitamin B11 and vitamin B12.

39. The method of claim 35, wherein the formulation further comprises calcium and magnesium.

40. The method of claim 39, wherein the calcium and magnesium are provided in a ratio of approximately 2 to 1 (2:1) of calcium to magnesium.

41. The method of claim 36, wherein the formulation further comprises calcium and magnesium.

42. The method of claim 41, wherein the calcium and magnesium are provided in a ratio of approximately 2 to 1 (2:1) of calcium to magnesium.

43. The method of claim 37, wherein the formulation further comprises calcium and magnesium.

44. The method of claim 43, wherein the calcium and magnesium are provided in a ratio of approximately 2 to 1 (2:1) of calcium to magnesium.

45. The method of claim 38, wherein the formulation further comprises calcium and magnesium.

46. The method of claim 45, wherein the calcium and magnesium are provided in a ratio of approximately 2 to 1 (2:1) of calcium to magnesium.

47. The method of claim 35, wherein the formulation further comprises a supplement selected from the group consisting of zinc, magnesium, and vitamin B6.

48. The method of claim 35, wherein the formulation further comprises kava kava.

49. The method of claim 35, wherein the compound is L-glutamine.

50. The method of claim 49, wherein the formulation further comprises kava kava.

51. The method of claim 35, wherein the disease is arthritis.

52. A method of increasing levels of s-adenosylmethionine within a subject's body, the method comprising administering to the subject a formulation comprising:
a first compound selected from the group consisting of L-methionine, betaine, and malic acid;
a second compound selected from the group consisting of L-methionine, betaine, and malic acid, wherein the second compound is different from the first; and
at least one co-factor.

53. The method of claim 52, wherein the formulation further comprises N-acetylcystein.

54. The method of claim 52, wherein the formulation further comprises at least one B-vitamin selected from the group consisting of vitamin B11 and vitamin B12.

55. The method of claim 53, wherein the formulation further comprises at least one B-vitamin selected from the group consisting of vitamin B11 and vitamin B12.

56. The method of claim 52, wherein the formulation further comprises calcium and magnesium.

57. The method of claim 56, wherein the calcium and magnesium are provided in a ratio of approximately 2 to 1 (2:1) of calcium to magnesium.

58. The method of claim 53, wherein the formulation further comprises calcium and magnesium.

59. The method of claim 58, wherein the calcium and magnesium are provided in a ratio of approximately 2 to 1 (2:1) of calcium to magnesium.

60. The method of claim 54, wherein the formulation further comprises calcium and magnesium.

61. The method of claim 60, wherein the calcium and magnesium are provided in a ratio of approximately 2 to 1 (2:1) of calcium to magnesium.

62. The method of claim 55, wherein the formulation further comprises calcium and magnesium.

63. The method of claim 62, wherein the calcium and magnesium are provided in a ratio of approximately 2 to 1 (2:1) of calcium to magnesium.

64. The method of claim 52, wherein the formulation further comprises a supplement selected from the group consisting of zinc, magnesium, and vitamin B6.

65. The method of claim 52, wherein the formulation further comprises kava kava.

66. The method of claim 52, further comprising L-glutamine.

67. The method of claim 66, wherein the formulation further comprises kava kava.

68. The method of claim 52, wherein the formulation is contained within a food stuff.
